(19) United States
(12) Patent Application Publication
(10) Pub. No.: US 2019/0282776 A1
(43) Pub. Date: Sep. 19, 2019

(54) ASYMMETRICAL-CUFFED ORAL OR NASAL ENDOTRACHEAL TUBE

(71) Applicants: INDUSTRY-ACADEMIC COOPERATION FOUNDATION, YONSEI UNIVERSITY, Seoul (KR); THE CATHOLIC UNIVERSITY OF KOREA INDUSTRY-ACADEMIC COOPERATION FOUNDATION, Seoul (KR)

(72) Inventors: Young-jun OH, Seoul (KR); Tae-dong KWEON, Seoul (KR); Young-min OH, Goyang-si (KR)

(73) Assignees: INDUSTRY-ACADEMIC COOPERATION FOUNDATION, YONSEI UNIVERSITY, Seoul (KR); THE CATHOLIC UNIVERSITY OF KOREA INDUSTRY-ACADEMIC COOPERATION FOUNDATION, Seoul (KR)

(21) Appl. No.: 16/301,329

(22) PCT Filed: May 12, 2017
(86) PCT No.: PCT/KR2017/004964

§ 371 Date: Nov. 13, 2018

(30) Foreign Application Priority Data
May 13, 2016 (KR) 10-2016-0058621
Apr. 27, 2017 (KR) 10-2017-0054658

(51) Int. Cl. A61M 16/04

(52) U.S. Cl. CPC A61M 16/0459; A61M 25/1011; A61M 16/0461; A61M 16/0418

(57) ABSTRACT

An oral or nasal endotracheal tube includes a tube part, a first balloon and a second balloon. The tube part extends along a direction with a tube shape and an end portion of the tube part is intubated into trachea. The first balloon extends with passing through the tube part, and expands or contracts a block cuff. The second balloon extends with passing through the tube part, and expands or contracts a guide cuff. The block cuff uniformly expands around the tube part and blocks the trachea after the tube part is intubated into the trachea. The guide cuff ununiformly expands around the tube part and guides the tube part to be intubated into the trachea.

### BACKGROUND

### 1. Field of Disclosure

The present disclosure of invention relates to an oral or nasal endotracheal tube, and more specifically the present disclosure of invention relates to an asymmetrical-cuffed oral or nasal endotracheal tube used for intubation through an oral cavity or a nasal cavity and guiding the endotracheal tube into a trachea more exactly.

### 2. Description of Related Technology

In the intubation, an oral intubation in which an endotracheal tube is intubated through an oral cavity is normally used, and for example, the intubation procedure using a laryngoscope, the procedure using a laryngeal mask airway and so on has been used.

However, when the oral or nasal intubation is hard to be performed in cases that cervical vertebrae of the patient is damaged, an oral cavity is closed due to temporomandibular joint spasticity, an injury or hemorrhage of the oral cavity occurs, tongue edema occurs and so on, a blind endotracheal intubation should be performed.

In case of the blind endotracheal intubation, as illustrated in FIG. 1 for the oral intubation or in FIG. 2 for the nasal intubation, an end portion 15 of a tube 11 is normally intubated into esophagus 3 not into trachea 2, and thus the intubation may be often failed.

### SUMMARY

The present invention is developed to solve the above-mentioned problems of the related arts. The present invention provides an oral or nasal endotracheal tube capable of guiding a tube into trachea to increase accuracy and probability of the intubation and to minimize an injury of the patient in case of the blind endotracheal intubation.

According to an example embodiment, an oral or nasal endotracheal tube includes a tube part, a first balloon and a second balloon. The tube part extends along a direction with a tube shape and an end portion of the tube part is intubated into trachea. The first balloon extends with passing through the tube part, and expands or contracts a block cuff. The second balloon extends with passing through the tube part, and expands or contracts a guide cuff. The block cuff uniformly expands around the tube part and blocks the trachea after the tube part is intubated into the trachea. The guide cuff ununiformly expands around the tube part and guides the tube part to be intubated into the trachea.

In an example embodiment, the second balloon may expand the guide cuff for the end portion of the tube part to be intubated into the trachea, and may contract the guide cuff after the tube part is intubated into the trachea. The first balloon may expand the block cuff to block the trachea after the tube part is intubated into the trachea.

In an example embodiment, the guide cuff may be disposed at a front side or a rear side of the block cuff.

In an example embodiment, the guide cuff may expand around the tube part with an asymmetric spherical shape, so that the tube part passes through a front side of a center of the guide cuff and an amount of the expansion of the guide cuff at a rear side of the tube part is larger than that at a front side of the tube part.

In an example embodiment, the guide cuff may be spaced apart from the end portion of the tube part by a predetermined distance when the guide cuff is disposed at the front side of the block cuff.

In an example embodiment, the guide cuff may expand with a hemispherical shape from the rear side of the tube part.

According to another example embodiment, an oral or nasal endotracheal tube includes a tube part and a balloon. The tube part extends along a direction with a tube shape, and an end portion of the tube part is intubated into trachea. The balloon extends with passing through the tube part, and expands or contracts a guide cuff. The guide cuff is spaced apart from the end portion of the tube by a predetermined position, ununiformly expands around the tube part to guide the tube part to be intubated into the trachea or to block the trachea after the tube part is intubated into the trachea.

In an example embodiment, the balloon may expand the guide cuff for the end portion of the tube part to be intubated into the trachea, and may contract the guide cuff after the tube part is intubated into the trachea.

In an example embodiment, the balloon may expand the guide cuff again to block the trachea after the tube part is intubated into the trachea.

In an example embodiment, when the tube part is intubated into the trachea, the tube part may be exchanged with an additional tube part having a block cuff after a tube exchanging catheter is inserted, so that the trachea is blocked.

In an example embodiment, the guide cuff may expand around the tube part with an asymmetric spherical shape, so that the tube part passes through a front side of a center of the guide cuff and an amount of the expansion of the guide cuff at a rear side of the tube part is larger than that at a front side of the tube part.

In an example embodiment, the guide cuff may expand with a hemispherical shape from a rear side of the tube part.

In an example embodiment, the guide cuff may include a first guide cuff expanding with a hemispherical shape from a front side of the tube part, and a second guide cuff expanding with a hemispherical shape from a rear side of the tube part. The balloon may include a first balloon expanding or contracting the first guide cuff, and a second balloon expanding or contracting the second guide cuff.

In an example embodiment, a volume of the first guide cuff may be less than that of the second guide cuff.

According to the present example embodiments, the guide cuff is added to the oral or nasal endotracheal tube in addition to the block cuff, and the guide cuff only expands from the rear side of the tube part to guide the tube part toward the trachea when the tube part is intubated through the oral or nasal cavity, so that the endotracheal intubation may be more correctly in case of the blind endotracheal intubation.

A portion of the guide cuff making contact with a rear side of pharynx expands to guide the tube part toward a front side of the pharynx, so that the tube part may be guided into the trachea in spite of the esophagus when the patient lie on a bed.

Here, the guide cuff may expand toward the rear side of the pharynx and toward the front side of the pharynx, and an amount of the expansion toward the rear side is larger than that toward the front side, so that the tube part is guided toward the front side of the pharynx but is not guided too much toward the front side of the pharynx. Thus, the guide tube may be guided into the trachea positioned in front of the esophagus more properly.

When the guide cuff is disposed at a rear side of the block cuff, the guide cuff may start to guide the tube part after the tube part is intubated into the esophagus. Thus, to solve the problem, the guide cuff is disposed at a front side of the block cuff and guides the tube part toward the trachea with a length of the tube part preceding the guide cuff maintained to be minimized. Accordingly, the tube part may be more correctly guided into the trachea.

Here, when the guide cuff is disposed at the front of the block cuff, the guide cuff expands with a hemispherical or asymmetric spherical shape and thus the tube part may be guided more effectively.

Further, with only the guide cuff, the guide cuff may guide the tube part into the trachea and may block the trachea after the tube part is intubated into the trachea. Thus, the guide cuff may effectively function both the guiding and the blocking without the block cuff

In addition, with only the guide cuff, the guide cuff may include first and second guide cuffs respectively expanding toward the front side and the rear side of the tube part, and thus the first and second guide cuffs may selectively expand considering the position of the tube part. Accordingly, the tube part may be guide into the trachea more correctly.

### BRIEF DESCRIPTION OF THE DRAWINGS

FIG. 1 is an anatomical diagram illustrating a conventional oral endotracheal tube intubated into esophagus;

FIG. 2 is an anatomical diagram illustrating a conventional nasal endotracheal tube intubated into esophagus;

FIG. 3 is a perspective view illustrating an oral or nasal endotracheal tube according to an example embodiment of the present invention;

FIG. 4 is a cross-sectional view taken along a line I-I′ of FIG. 3;

FIG. 5 is an anatomical diagram illustrating the oral or nasal endotracheal tube of FIGS. 3 and 4, intubated through an oral cavity into trachea;

FIG. 6 is an anatomical diagram illustrating the oral or nasal endotracheal tube of FIGS. 3 and 4, intubated through a nasal cavity into trachea;

FIG. 7 is a perspective view illustrating an oral or nasal endotracheal tube according to another example embodiment of the present invention;

FIG. 8 is a cross-sectional view taken along a line II-II′ of FIG. 7;

FIG. 9 is an anatomical diagram illustrating the oral or nasal endotracheal tube of FIGS. 7 and 8, guided by a guide cuff;

FIG. 10 is an anatomical diagram illustrating the oral or nasal endotracheal tube of FIGS. 7 and 8, blocked by a block cuff;

FIG. 11 is a perspective view illustrating an oral or nasal endotracheal tube according to still another example embodiment of the present invention;

FIG. 12 is a perspective view illustrating an oral or nasal endotracheal tube according to still another example embodiment of the present invention;

FIG. 13 is a cross-sectional view taken along a line III-III′ of FIG. 12;

FIG. 14 is an anatomical diagram illustrating the oral or nasal endotracheal tube of FIGS. 12 and 13, guided by a guide cuff;

FIG. 15 is an anatomical diagram illustrating the oral or nasal endotracheal tube of FIGS. 12 and 13, intubated into trachea;

FIG. 16 is an anatomical diagram illustrating the oral or nasal endotracheal tube of FIGS. 12 and 13, blocked by the guide cuff;

FIG. 17 is a perspective view illustrating an oral or nasal endotracheal tube according to still another example embodiment of the present invention; and

FIG. 18 is a cross-sectional view taken along a line IV-IV′ of FIG. 17.

### REFERENCE NUMERALS

100, 200, 300, 400: oral or nasal endotracheal tube
110, 210, 310, 410: tube part
120, 220, 420: first balloon
121, 221, 421: first inner tube
130, 230, 330, 430: second balloon
131, 231, 331, 431: second inner tube
140, 240: block cuff
150, 250, 350: guide cuff
440: first guide cuff
450: second guide cuff

### DETAILED DESCRIPTION

The invention is described more fully hereinafter with reference to the accompanying drawings, in which embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. In the drawings, the size and relative sizes of layers and regions may be exaggerated for clarity.

The terminology used herein is for the purpose of describing particular embodiments only and is not intended to be limiting of the invention. As used herein, the singular forms “a,” “an” and “the” are intended to include the plural forms as well, unless the context clearly indicates otherwise.

It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

Hereinafter, exemplary embodiment of the invention will be explained in detail with reference to the accompanying drawings.

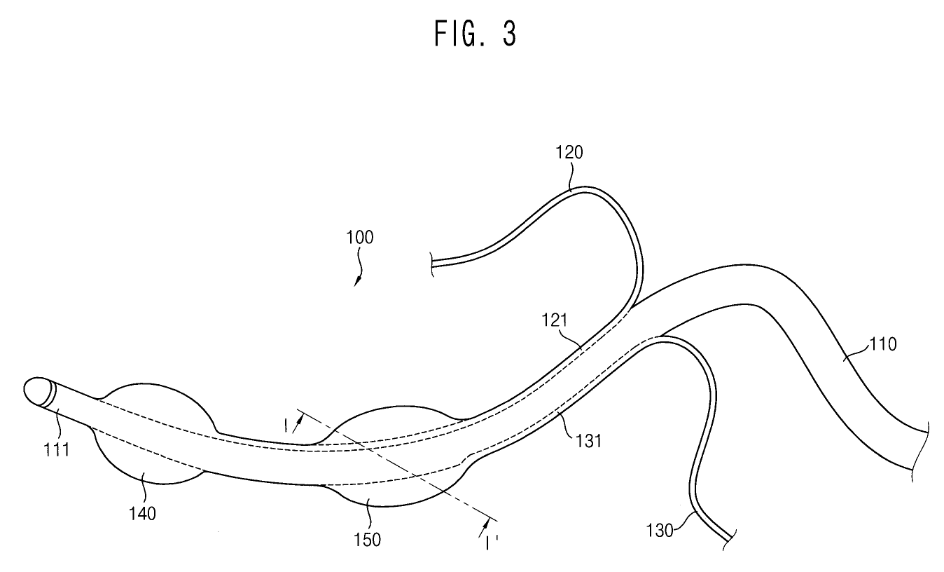

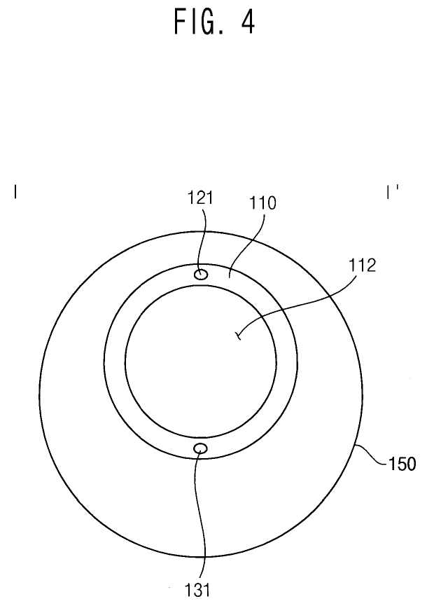

FIG. 3 is a perspective view illustrating an oral or nasal endotracheal tube according to an example embodiment of the present invention. FIG. 4 is a cross-sectional view taken along a line I-I′ of FIG. 3.

Referring to FIGS. 3 and 4, the oral or nasal endotracheal tube according to the present example embodiment includes a tube part 110, a first balloon 120, a second balloon 130, a block cuff 140 and a guide cuff 150.

The tube part 110 includes a flexible material, and extends along a direction. The tube part 110 is a tube having an opening at a center through which an air passes.

The first balloon 120 passes through a side of the tube part 110, and is connected to the block cuff 140.

The first balloon 120 includes a first inner tube 121, and the first inner tube 121 passes through the side of the tube part 110 and is connected to the block cuff 140 positioned at an end portion 111 of the tube part 110.

Thus, the block cuff 140 expands when the air or the fluid is flowed into the first balloon 120, and contracts when the air or the fluid is flowed out from the first balloon 120.

Here, the first inner tube 121 may pass through a wall of the tube 100 as illustrated in FIG. 3.

The second balloon 130 passes through an opposite side of the tube part 110, and is connected to the guide cuff 150.

The second balloon 130 includes a second inner tube 131, and the second inner tube 131 passes though the opposite side of the tube part 110 and is connected to the guide cuff 150 positioned at a rear side of the block cuff 140 in the tube part 110.

Thus, the guide cuff 150 expands when the air or the fluid is flowed into the second balloon 130, and contracts when the air or the fluid is flowed out from the second balloon 130.

Here, the second inner tube 131 may pass through a wall of the tube 100 as illustrated in FIG. 3, and may be disposed opposite to the first inner tube 121.

The guide cuff 150 is connected to the rear side of the tube part 110, for example a lower side of the tube part 110 as illustrated in the figure, so that the second inner tube 131 may pass through a lower side wall of the tube part 110 as illustrated in the figure.

The block cuff 140 is connected to the end portion 111 of the tube part 110, and expands or contracts as the air or the fluid is flowed in or out from the first balloon 120.

Here, the block cuff 140 is spaced apart from an end of the tube part 110 by a predetermined distance, and thus the end portion 111 of the tube part 110 is extended from the block cuff 140 by a predetermined distance.

The block cuff 140 surrounds the tube part 110, and thus expands uniformly around the tube part 110 when expanding.

Thus, the block cuff 140 uniformly blocks the trachea with intubated into the trachea when the block cuff 140 expands.

The guide cuff 150 is disposed at a rear side of the block cuff 140, and for example, considering an average size of the patient, may be disposed at the rear side of the block cuff 140 by less than about 5 cm.

The guide cuff 150 expands or contracts as the air or the fluid is flowed in or out from the second balloon 130.

The guide cuff 150 ununiformly or asymmetrically expands around the tube part 110, as illustrated in FIG. 4.

The guide cuff 150 expands around the tube part 110 with an asymmetric spherical shape, and thus the tube part 110 passes through a front side of a center of the guide cuff 150 (as illustrated in FIG. 4, an upper side of the center of the guide cuff 150). Thus, an amount of expanding volume of the guide cuff 150 toward the rear side of the tube part 110 is larger than that toward the front side of the tube part 110

For example, as illustrated in FIG. 4, a cross-sectional shape of the guide cuff 150 may be an asymmetric donut shape.

Here, a portion of the guide cuff 150 expanding toward the rear side of the tube part 110 guides the tube part 110 forwardly, and a portion of the guide cuff 150 expanding toward the front side of the tube part 110 guides the tube part 110 backwardly.

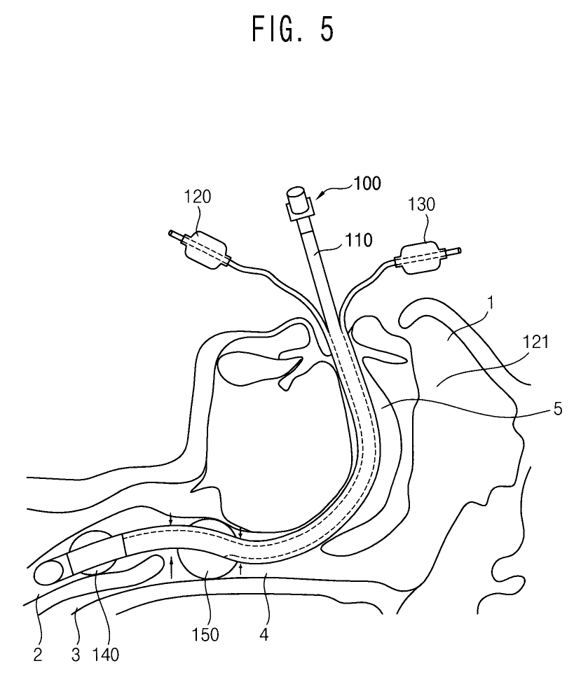

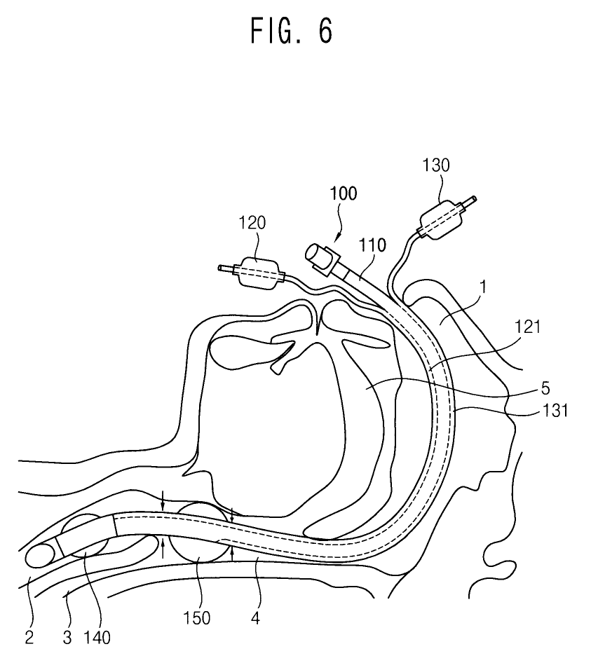

FIG. 5 is an anatomical diagram illustrating the oral or nasal endotracheal tube of FIGS. 3 and 4, intubated through an oral cavity into trachea. FIG. 6 is an anatomical diagram illustrating the oral or nasal endotracheal tube of FIGS. 3 and 4, intubated through a nasal cavity into trachea.

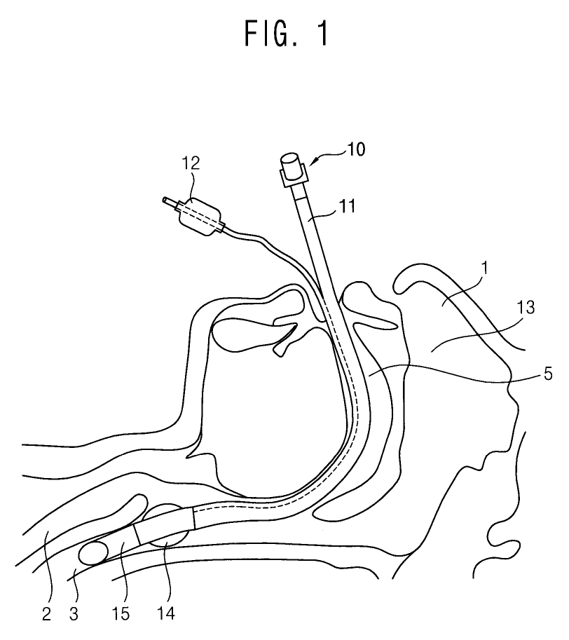

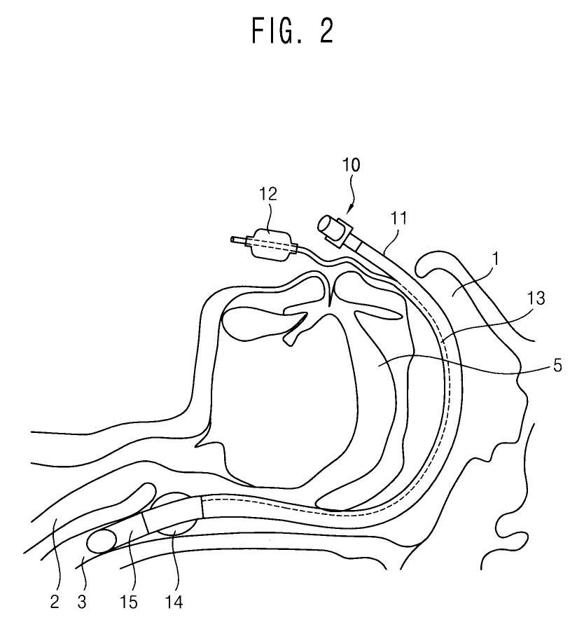

As illustrated in FIGS. 1 and 2, in the blind endotracheal intubation using the conventional oral or nasal endotracheal tube 10, the esophagus 3 is positioned at the rear side of the trachea 2 when the patient lie on a bed (the esophagus 3 is positioned at the lower side of the trachea 2 as illustrated in the figure, but the esophagus 3 is positioned at the rear side of the trachea 2 when the patient stands, and thus the positions of the elements of the example embodiments of the present invention will be explained based on the standing position of the patient), and thus the end portion 15 of the tube 10 is easy to be intubated into the esophagus 3 without the guiding. Thus, the blind endotracheal intubation may be performed more incorrectly.

Thus, in the present example embodiment, the above mentioned problem may be solved. As illustrated in FIGS. 5 and 6, when the tube part 110 passes through the pharynx 4 via the oral or nasal cavity, the guide cuff 150 guides the end portion 111 of the tube part 110 forwardly with expanding toward the rear side of the tube part 110, and guides the end portion 111 of the tube part 110 backwardly with expanding toward the front side of the tube part 110.

Here, an expanding volume of the guide cuff 150 at the rear side from the tube part 110 is larger than that at the front side from the tube part 110, and thus the tube part 110 is guided forwardly. In this case, when the guide cuff 150 only expands at the rear side from the tube part 110, the tube part 110 is only guided forwardly. Thus, the tube part 110 may make contact with a front side of the pharynx 4 when the tube part 110 is too much guided forwardly.

Thus, as in the present example embodiment, the guide cuff 150 may expand at the front side from the tube part 110 even though the expanding volume is not too much, and thus the patient may be more comfortable and the tube part 110 may be guided into the trachea 2 more effectively.

When the patient lies on the bed, the esophagus 3 is disposed at the rear side of the trachea 2 and thus the end portion 111 of the tube part 110 guided forwardly may be intubated into the trachea 2 more easily. Thus, the endotracheal intubation may be performed more correctly.

Then, when the end portion 111 of the tube part 110 is intubated into the trachea 2, the second balloon 130 contracts the guide cuff 150.

However, the first balloon 120 expands the block cuff 140 to block the trachea 2, and finish the endotracheal intubation.

Although not shown in the figure, the guide cuff 150 may have a hemispherical shape only expanding to a rear side of the tube part 110.

When the tube part 110 passes through the pharynx 4 via the oral or nasal cavity and the second balloon 130 expands the guide cuff 150, the guide cuff 150 expands only at the rear side of the tube part 110 and guides the end portion 111 of the tube part 110 forwardly, and thus the tube part 110 is guided into the trachea 2.

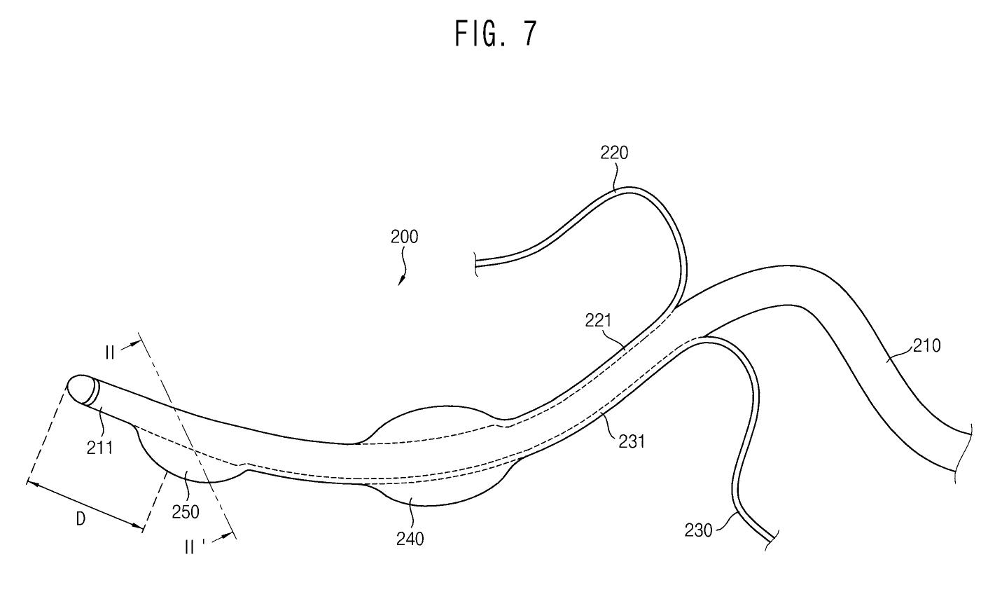

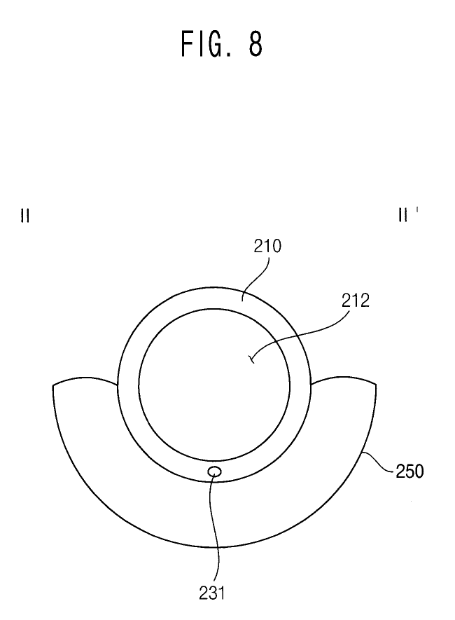

FIG. 7 is a perspective view illustrating an oral or nasal endotracheal tube according to another example embodiment of the present invention. FIG. 8 is a cross-sectional view taken along a line II-II′ of FIG. 7.

Referring to FIGS. 7 and 8, the oral or nasal endotracheal tube 200 according to the present example embodiment includes a tube part 210, a first balloon 220, a second balloon 230, a block cuff 240 and a guide cuff 250.

The tube part 210 includes a flexible material, and extends along a direction. The tube part 210 is a tube having an opening at a center through which an air passes.

The first balloon 220 is connected to the block cuff 240 via a first inner tube 221 passing through a side of the tube part 210. Here, a structure and a movement of the first balloon 220, the first inner tube 221 and the block cuff 240 are substantially same as those of the first balloon 120, the first inner tube 121 and the block cuff 140, except that the block cuff 240 is disposed at a rear side of the guide cuff 250, and thus any repetitive explanation will be omitted.

Likewise, the second balloon 230 is connected to the guide cuff 250 via a second inner tube 231 passing through an opposite side of the tube part 210. Here, a structure and a movement of the second balloon 230, the second inner tube 231 and the guide cuff 250 are substantially same as those of the second balloon 130, the second inner tube 131 and the guide cuff 150, except that the block cuff 240 is disposed at a rear side of the guide cuff 250 and the guide cuff 250 expands with a hemispherical shape, and thus any repetitive explanation will be omitted.

In the present example embodiment, the guide cuff 250 is disposed at a front side of the block cuff 240, and for example, considering an average size of the patient, a distance D between the guide cuff 250 and the end portion 211 of the tube part 210 may be between about 1 cm and about 5 cm.

The guide cuff 250 is spaced apart from the end portion 211 of the tube part 210 by the distance D, not disposed at the end portion 211 of the tube part 210, and thus the end portion 211 may be more effectively guided into the trachea 2.

The guide cuff 250 expands only toward the rear side of the tube part 210 with a hemispherical shape.

Thus, as illustrated in FIG. 8, a cross-sectional shape of the guide cuff 250 may be a half-cut donut shape.

Accordingly, the guide cuff 250 expands only toward the rear side of the tube part 210, and thus the tube part 210 is guided forwardly to intubate the tube part 210 into the trachea 2.

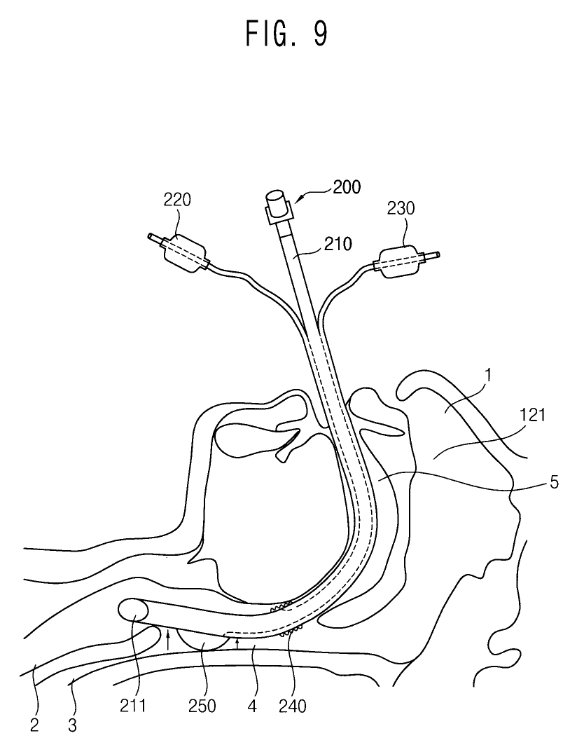

FIG. 9 is an anatomical diagram illustrating the oral or nasal endotracheal tube of FIGS. 7 and 8, guided by a guide cuff.

Referring to FIG. 9, when the endotracheal intubation using the oral or nasal endotracheal tube 200 of the present example embodiment, firstly, the guide cuff 250 expands with the block cuff 240 contracted.

As the guide cuff 250 expands, the tube part 210 is guided to the front side of the pharynx 4 from the back side of the pharynx 4. Thus, the end portion 211 of the tube part 210 is intubated into the trachea 2 relatively forwardly positioned not into the esophagus 3 relatively backwardly positioned.

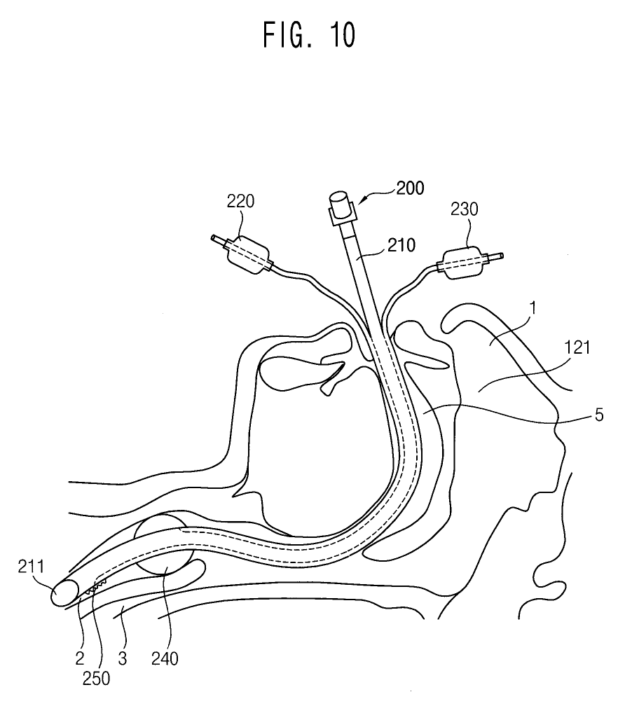

FIG. 10 is an anatomical diagram illustrating the oral or nasal endotracheal tube of FIGS. 7 and 8, blocked by a block cuff.

Referring to FIG. 10, the guide cuff 250 expands to guide the end portion 211 of the tube part 210 into the trachea 2, and then the guide cuff 250 is contracted and the tube part 210 is more intubated into the trachea 2.

Then, the block cuff 240 expands to block the trachea 2, and then the endotracheal intubation is finished.

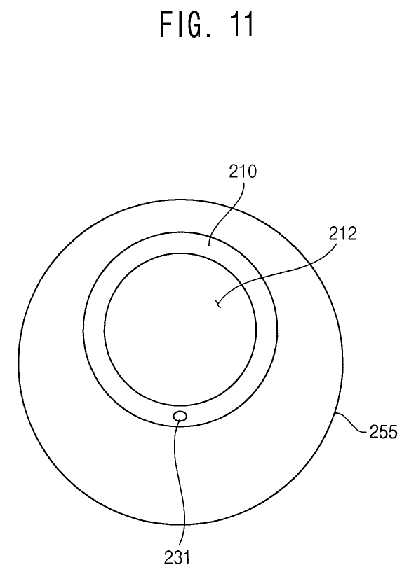

FIG. 11 is a perspective view illustrating an oral or nasal endotracheal tube according to still another example embodiment of the present invention.

The oral or nasal endotracheal tube 200 according to the present example embodiment is substantially same as the oral or nasal endotracheal tube 200 explained referring to FIG. 7, except for a shape of a guide cuff 255, and thus same reference numerals are used for same elements and any repetitive explanation will be omitted.

Referring to FIG. 11, in the oral or nasal endotracheal tube 200 according to the present example embodiment, the guide cuff 255 has the shape same as the guide cuff 150 of the oral or nasal endotracheal tube 100 explained referring to FIGS. 3 and 4.

The guide cuff 255 expands around the tube part 210, but as illustrated in FIG. 11, the guide cuff 255 ununiformly and asymmetrically expands around the tube part 210.

The guide cuff 255 asymmetrically expands around the tube part 210, and thus the tube part 210 passes through the front side of the center of the guide cuff 255. Thus, an expanding volume of the guide cuff 255 toward the rear side from the tube part 210 is larger than that toward the front side from the tube part 210.

For example, as illustrated in FIG. 11, the cross-sectional shape of the guide cuff 255 may be an asymmetric donut shape.

Here, a portion of the guide cuff 255 expanding toward the rear side of the tube part 210 guides the tube part 210 forwardly, and a portion of the guide cuff 255 expanding toward the front side of the tube part 210 guides the tube part 210 backwardly.

Here, the guiding method and the effect of the tube part 210 as the guide cuff 255 asymmetrically expands, is substantially same as those explained referring to FIGS. 3 and 4, and thus any repetitive explanation will be omitted.

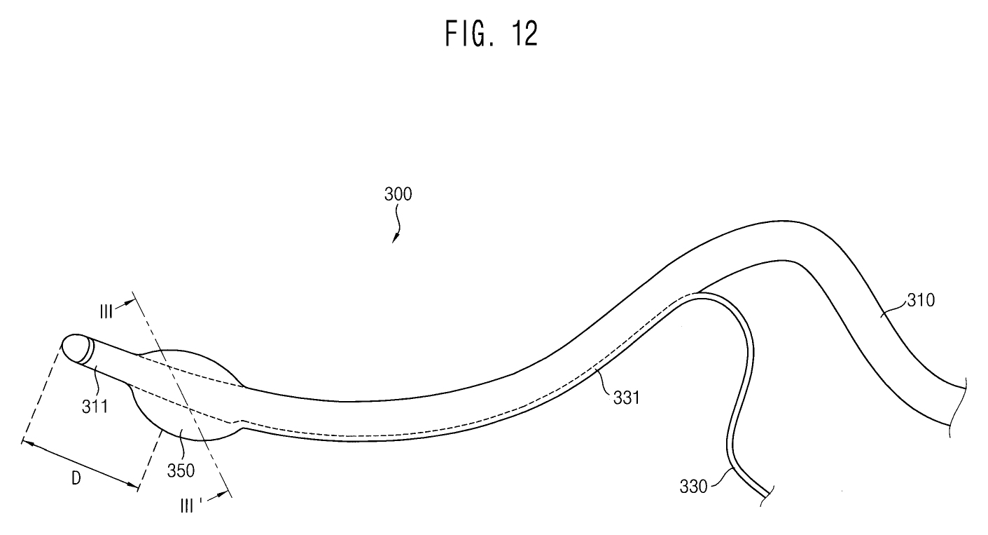

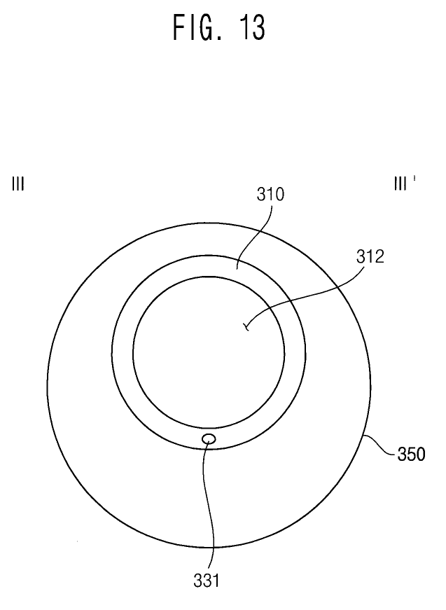

FIG. 12 is a perspective view illustrating an oral or nasal endotracheal tube according to still another example embodiment of the present invention. FIG. 13 is a cross-sectional view taken along a line III-III′ of FIG. 12.

Referring to FIGS. 12 and 13, the oral or nasal endotracheal tube 300 according to the present example embodiment includes a tube part 310, a second balloon 330 and a guide cuff 350.

The tube part 310 includes a flexible material, and extends along a direction. The tube part 310 is a tube having an opening at a center through which an air passes.

The second balloon 330 passes through a side of the tube part 310, and is connected to the guide cuff 350.

The second balloon 330 includes a second inner tube 331, and the second inner tube 331 passes through the side of the tube part 310 and is connected to the guide cuff 350.

Thus, the guide cuff 350 expands when the air or the fluid is flowed into the second balloon 330, and contracts when the air or the fluid is flowed out from the second balloon 330.

In the present example embodiment, the guide cuff 350 is only disposed at a front side of the tube part 310 without the block cuff, and for example, considering an average size of the patient, a distance D between the guide cuff 350 and the end portion 311 of the tube part 310 may be between about 1 cm and about 5 cm.

The guide cuff 350 is spaced apart from the end portion 311 of the tube part 310 by the distance D, not disposed at the end portion 311 of the tube part 310, and thus the end portion 311 may be more effectively guided into the trachea 2.

The guide cuff 350 according to the present example embodiment has a shape substantially same as that of the guide cuff 150 explained referring to FIGS. 3 and 4.

The guide cuff 350 expands around the tube part 310, but as illustrated in FIG. 13, the guide cuff 350 ununiformly and asymmetrically expands around the tube part 310.

The guide cuff 350 asymmetrically expands around the tube part 310, and thus the tube part 310 passes through the front side of the center of the guide cuff 350. Thus, an expanding volume of the guide cuff 350 toward the rear side from the tube part 310 is larger than that toward the front side from the tube part 310.

For example, as illustrated in FIG. 13, the cross-sectional shape of the guide cuff 350 may be an asymmetric donut shape.

Here, a portion of the guide cuff 350 expanding toward the rear side of the tube part 310 guides the tube part 310 forwardly, and a portion of the guide cuff 350 expanding toward the front side of the tube part 310 guides the tube part 310 backwardly.

Here, the guiding method and the effect of the tube part 310 as the guide cuff 350 asymmetrically expands, is substantially same as those explained referring to FIGS. 3 and 4, and thus any repetitive explanation will be omitted.

Further, in the present example embodiment, the guide cuff 350 also blocks the trachea 2 after the tube part 310 is intubated into the trachea 2, in addition to the guiding the tube part 310.

Here, the guide cuff 350 asymmetrically expands, and thus the tube part 310 inside of the trachea 2 is positioned at a side of a center of the trachea 2 but the guide cuff 350 may block the trachea 2.

Although not shown in the figure, if the tube part 310 should be positioned at the center of the trachea 2, after the tube part 310 is intubated into the trachea 2, the oral or nasal endotracheal tube 300 having the guide cuff 350 may be exchanged into a normal oral or nasal endotracheal tube having the block cuff with inserting a tube exchanging catheter and then the trachea 2 is blocked by the block cuff.

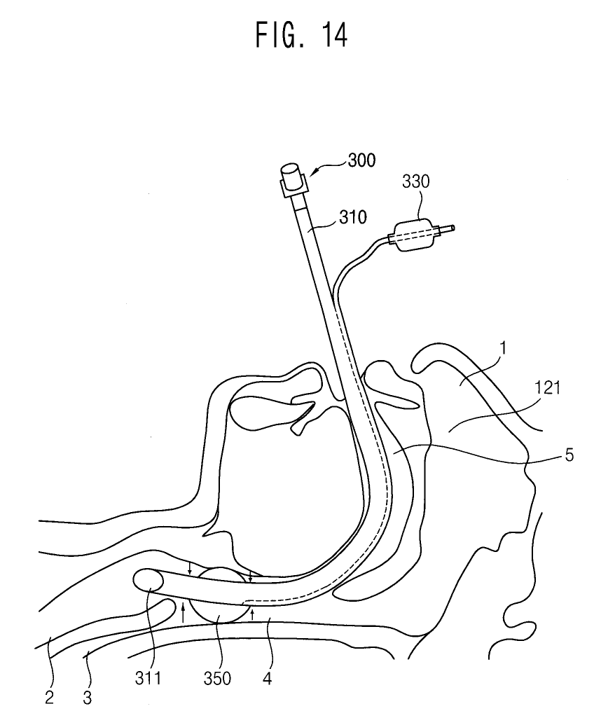

FIG. 14 is an anatomical diagram illustrating the oral or nasal endotracheal tube of FIGS. 12 and 13, guided by a guide cuff.

Referring to FIG. 14, when the endotracheal intubation is performed using the oral or nasal endotracheal tube 300, firstly, the guide cuff 350 expands to guide the end portion 311 of the tube part 310 into the trachea 2.

The guide cuff 350 asymmetrically expands and the rear side of the tube part 310 expands more than the front side of the tube part 310, and thus the end portion 311 of the tube part 310 is guided forwardly in the pharynx 4. Thus, the end portion 311 is intubated into the trachea 2 positioned at the front side of the esophagus 3.

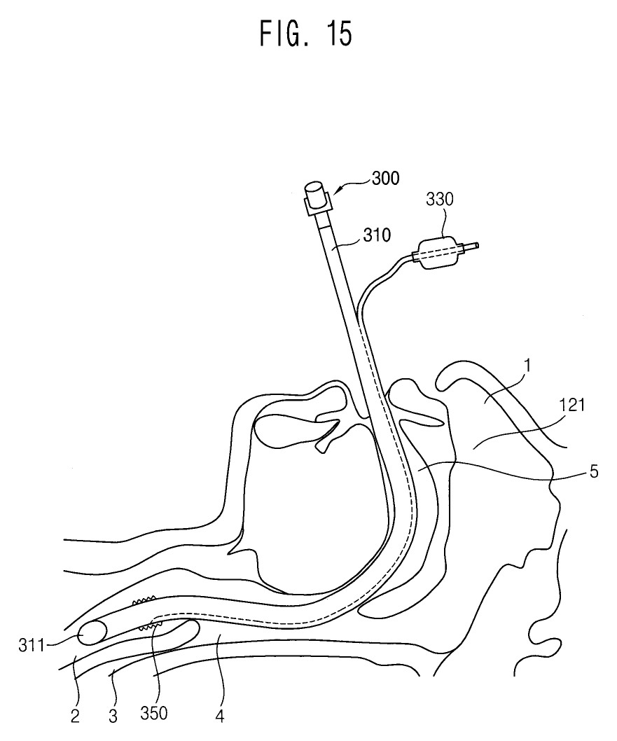

FIG. 15 is an anatomical diagram illustrating the oral or nasal endotracheal tube of FIGS. 12 and 13, intubated into trachea.

Referring to FIG. 15, after the end portion 311 of the tube part 310 is intubated into the trachea 2 by the guide cuff 350, the guide cuff 350 is contracted and the tube part 310 is continuously intubated into the trachea 2. Thus, the tube part 310 is fully intubated.

Here, the tube part 310 is fully intubated such that the contracted guide cuff 350 is positioned inside of the trachea 2.

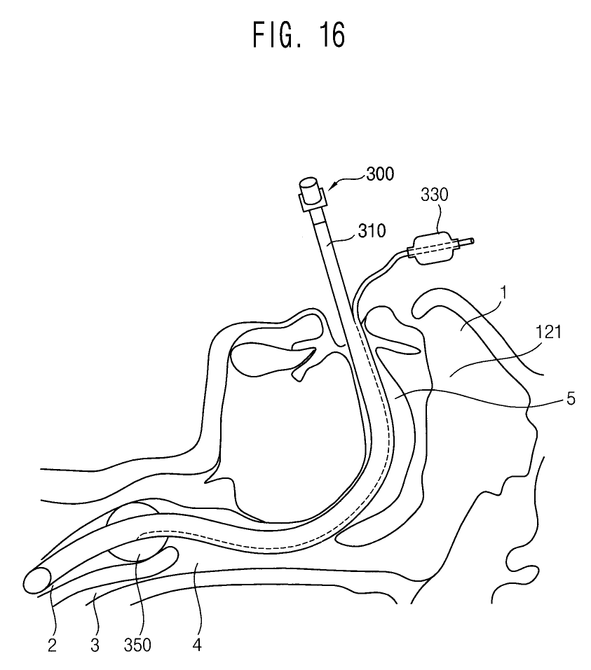

FIG. 16 is an anatomical diagram illustrating the oral or nasal endotracheal tube of FIGS. 12 and 13, blocked by the guide cuff.

Referring to FIG. 16, then, the guide cuff 350 expands again, and thus the trachea 2 is blocked.

Here, even though the guide cuff 350 asymmetrically expands, the trachea 2 may be fully blocked, and thus the endotracheal intubation is finished.

Alternatively, although not shown in the figure, with the oral or nasal endotracheal tube 300 having the guide cuff 350 intubated into the trachea 2, the oral or nasal endotracheal tube 300 having the guide cuff 350 may be exchanged into a normal oral or nasal endotracheal tube having the block cuff with inserting a tube exchanging catheter and then the trachea 2 is blocked by the block cuff.

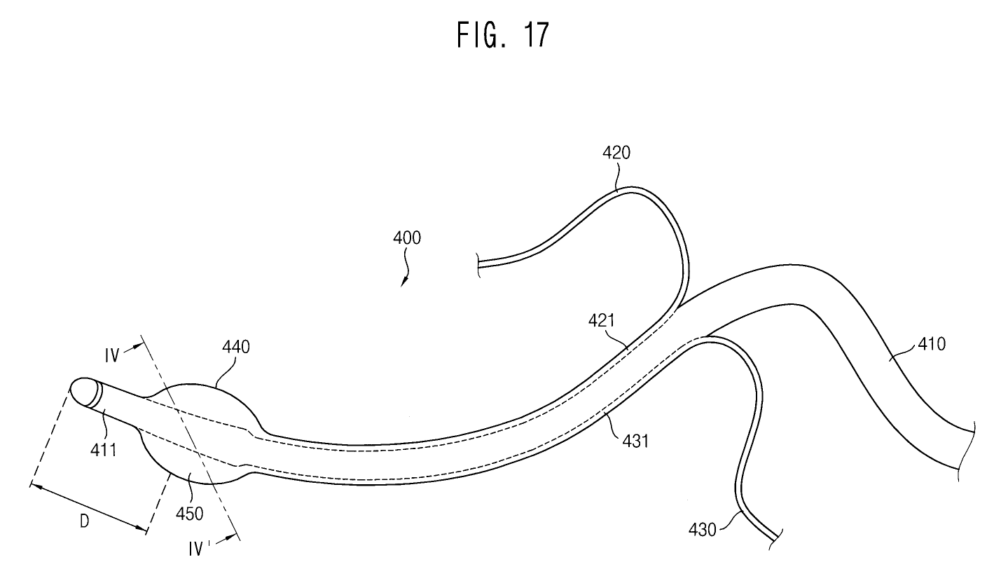

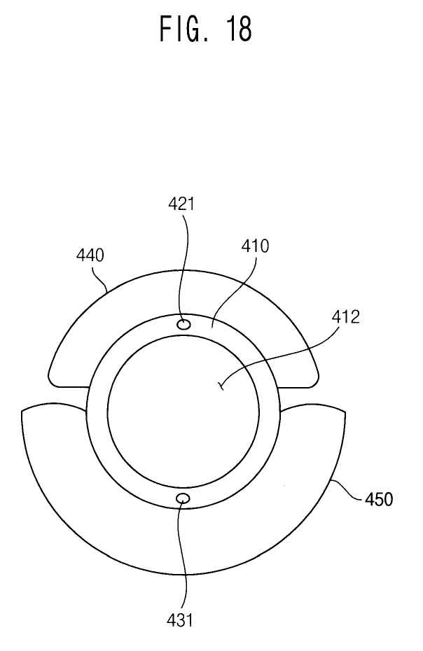

FIG. 17 is a perspective view illustrating an oral or nasal endotracheal tube according to still another example embodiment of the present invention. FIG. 18 is a cross-sectional view taken along a line IV-IV′ of FIG. 17.

Referring to FIGS. 17 and 18, the oral or nasal endotracheal tube 400 according to the present example embodiment includes a tube part 410, a first balloon 420, a second balloon 430, a first guide cuff 440 and a second guide cuff 450.

The tube part 410 includes a flexible material, and extends along a direction. The tube part 110 is a tube having an opening at a center through which an air passes.

The first balloon 420 passes through a side of the tube part 410, and is connected to the first guide cuff 440.

The first balloon 420 includes a first inner tube 421, and the first inner tube 421 passes through the side of the tube part 410 and is connected to the first guide cuff 440.

Thus, the first guide cuff 440 expands when the air or the fluid is flowed into the first balloon 420, and contracts when the air or the fluid is flowed out from the first balloon 420.

The second balloon 430 passes through an opposite side of the tube part 410, and is connected to the second guide cuff 450.

The second balloon 430 includes a second inner tube 431, and the second inner tube 431 passes though the opposite side of the tube part 410 and is connected to the second guide cuff 450.

Thus, the second guide cuff 450 expands when the air or the fluid is flowed into the second balloon 430, and contracts when the air or the fluid is flowed out from the second balloon 430.

In the present example embodiment, the first and second guide cuffs 440 and 450 are disposed at a front side of the tube part 410, and for example, considering an average size of the patient, a distance D between the first and second guide cuffs 440 and 450 and the end portion 411 of the tube part 410 may be between about 1 cm and about 5 cm.

The first and second guide cuffs 440 and 450 are spaced apart from the end portion 411 of the tube part 410 by the distance D, not disposed at the end portion 411 of the tube part 410, and thus the end portion 411 may be more effectively guided into the trachea 2.

Here, the first guide cuff 440 expands at the front side of the tube part 410 and has a hemispherical shape. The second guide cuff 450 expands at the rear side of the tube part 410 and has a hemispherical shape.

As illustrated in FIG. 18, a cross-sectional shape of the first guide cuff 440 may be a half-cut donut shape, and expands only toward the front side of the tube part 410 with the hemispherical shape. A cross-sectional shape of the second guide cuff 440 may be a half-cut donut shape, and expands only toward the rear side of the tube part 410 with the hemispherical shape.

Accordingly, the second guide cuff 450 expands only toward the rear side of the tube part 410, and thus the tube part 410 is guided forwardly. The first guide cuff 440 expands only toward the front side of the tube part 410, and thus the tube part 410 is guided backwardly.

Here, a size or a volume of the expanded hemispherical shape of the first guide cuff 440 is smaller or less than that of the second guide cuff 450.

Since the expanded volumes of the first and second guide cuffs 440 and 450 are different from each other, the tube part 410 may be guided into the trachea 2 more effectively considering the position of the tube part 410 or the physical structure of the patient. Here, the guiding method and the effect of the tube part 410 as the first and second guide cuffs 440 and 450 asymmetrically expand, is substantially same as those explained referring to FIGS. 3 and 4, and thus any repetitive explanation will be omitted.

Further, in the present example embodiment, the first and second guide cuffs 440 and 450 expands to block the trachea 2 after the tube part 410 is intubated into the trachea 2, in addition to guiding the tube part 410.

Here, the first and second guide cuffs 440 and 450 asymmetrically expand to be positioned at a side of the center of the tube part 410, but the first and second guide cuffs 440 and 450 may block the inside of the trachea 2.

Although not shown in the figure, if the tube part 410 should be positioned at the center of the trachea 2, after the tube part 410 is intubated into the trachea 2, the oral or nasal endotracheal tube 400 having the first and second guide cuffs 440 and 450 may be exchanged into a normal oral or nasal endotracheal tube having the block cuff with inserting a tube exchanging catheter and then the trachea 2 is blocked by the block cuff.

Further, the first and second guide cuffs 440 and 450 are individually controlled by the first and second balloons 420 and 430, and thus the first and second guide cuffs 440 and 450 may be selectively or simultaneously controlled to be expanded or contracted. Thus, the tube part 410 may be guided more effectively.

For example, when the tube part 410 does not make contact with the front side of the pharynx 4 so as not to cause inconvenience to the patient and guides the tube part 410 effectively, with only the second guide cuff 450 expanding, the endotracheal intubation may be performed with the first guide cuff 440 being contracted.

Further, the first and second guide cuffs 440 and 450 may be expanded or contracted with various kinds of combination, considering the relative position of the trachea 2 and the esophagus 3 in each patient.

The specific intubation methods using the oral or nasal endotracheal tube 400 of the present example embodiment are substantially same as those using the oral or nasal endotracheal tube 300 explained referring to FIGS. 14 to 16, except that the first and second guide cuffs 440 and 450 are individually and selectively controlled to be expanded or contracted, and thus any repetitive explanation is omitted.

According to the example embodiments, the guide cuff is added to the oral or nasal endotracheal tube in addition to the block cuff, and the guide cuff only expands from the rear side of the tube part to guide the tube part toward the trachea when the tube part is intubated through the oral or nasal cavity, so that the endotracheal intubation may be more correctly in case of the blind endotracheal intubation.

A portion of the guide cuff making contact with a rear side of pharynx expands to guide the tube part toward a front side of the pharynx, so that the tube part may be guided into the trachea in spite of the esophagus when the patient lie on a bed.

Here, the guide cuff may expand toward the rear side of the pharynx and toward the front side of the pharynx, and an amount of the expansion toward the rear side is larger than that toward the front side, so that the tube part is guided toward the front side of the pharynx but is not guided too much toward the front side of the pharynx. Thus, the guide tube may be guided into the trachea positioned in front of the esophagus more properly.

When the guide cuff is disposed at a rear side of the block cuff, the guide cuff may start to guide the tube part after the tube part is intubated into the esophagus. Thus, to solve the problem, the guide cuff is disposed at a front side of the block cuff and guides the tube part toward the trachea with a length of the tube part preceding the guide cuff maintained to be minimized. Accordingly, the tube part may be more correctly guided into the trachea.

Here, when the guide cuff is disposed at the front of the block cuff, the guide cuff expands with a hemispherical or asymmetric spherical shape and thus the tube part may be guided more effectively.

Further, with only the guide cuff, the guide cuff may guide the tube part into the trachea and may block the trachea after the tube part is intubated into the trachea. Thus, the guide cuff may effectively function both the guiding and the blocking without the block cuff

In addition, with only the guide cuff, the guide cuff may include first and second guide cuffs respectively expanding toward the front side and the rear side of the tube part, and thus the first and second guide cuffs may selectively expand considering the position of the tube part. Accordingly, the tube part may be guide into the trachea more correctly.

Having described the example embodiments of the present invention and its advantage, it is noted that various changes, substitutions and alterations can be made herein without departing from the spirit and scope of the invention as defined by appended claims.

What is claimed is:

1. An oral or nasal endotracheal tube comprising:
a tube part extending along a direction with a tube shape, an end portion of the tube part being intubated into trachea;
a first balloon extending with passing through the tube part, expanding or contracting a block cuff; and
a second balloon extending with passing through the tube part, expanding or contracting a guide cuff,
wherein the block cuff uniformly expands around the tube part and blocks the trachea after the tube part is intubated into the trachea,
wherein the guide cuff ununiformly expands around the tube part and guides the tube part to be intubated into the trachea.

2. The oral or nasal endotracheal tube of claim 1, wherein the second balloon expands the guide cuff for the end portion of the tube part to be intubated into the trachea, and contracts the guide cuff after the tube part is intubated into the trachea,
wherein the first balloon expands the block cuff to block the trachea after the tube part is intubated into the trachea.

3. The oral or nasal endotracheal tube of claim 2, wherein the guide cuff is disposed at a front side or a rear side of the block cuff.

4. The oral or nasal endotracheal tube of claim 3, wherein the guide cuff expands around the tube part with an asymmetric spherical shape, so that the tube part passes through a front side of a center of the guide cuff and an amount of the expansion of the guide cuff at a rear side of the tube part is larger than that at a front side of the tube part.

5. The oral or nasal endotracheal tube of claim 3, wherein the guide cuff is spaced apart from the end portion of the tube part by a predetermined distance when the guide cuff is disposed at the front side of the block cuff.

6. The oral or nasal endotracheal tube of claim 3, wherein the guide cuff expands with a hemispherical shape from the rear side of the tube part.

7. An oral or nasal endotracheal tube comprising:
a tube part extending along a direction with a tube shape, an end portion of the tube part being intubated into trachea; and
a balloon extending with passing through the tube part, expanding or contracting a guide cuff,
wherein the guide cuff is spaced apart from the end portion of the tube by a predetermined position, ununiformly expands around the tube part to guide the tube part to be intubated into the trachea or to block the trachea after the tube part is intubated into the trachea.

8. The oral or nasal endotracheal tube of claim 7, wherein the balloon expands the guide cuff for the end portion of the tube part to be intubated into the trachea, and contracts the guide cuff after the tube part is intubated into the trachea.

9. The oral or nasal endotracheal tube of claim 8, wherein the balloon expands the guide cuff again to block the trachea after the tube part is intubated into the trachea.

10. The oral or nasal endotracheal tube of claim 8, wherein when the tube part is intubated into the trachea, the tube part is exchanged with an additional tube part having a block cuff after a tube exchanging catheter is inserted, so that the trachea is blocked.

11. The oral or nasal endotracheal tube of claim 8, wherein the guide cuff expands around the tube part with an asymmetric spherical shape, so that the tube part passes through a front side of a center of the guide cuff and an amount of the expansion of the guide cuff at a rear side of the tube part is larger than that at a front side of the tube part.

12. The oral or nasal endotracheal tube of claim 8, wherein the guide cuff expands with a hemispherical shape from a rear side of the tube part.

13. The oral or nasal endotracheal tube of claim 8, wherein the guide cuff comprises:
a first guide cuff expanding with a hemispherical shape from a front side of the tube part; and
a second guide cuff expanding with a hemispherical shape from a rear side of the tube part,
wherein the balloon comprises:
a first balloon expanding or contracting the first guide cuff; and
a second balloon expanding or contracting the second guide cuff.

14. The oral or nasal endotracheal tube of claim 13, wherein a volume of the first guide cuff is less than that of the second guide cuff.
